(19) United States
(12) Patent Application Publication
(10) Pub. No.: US 2010/0280035 A1
(43) Pub. Date: Nov. 4, 2010

(54) SOLID PHARMACEUTICAL COMPOSITION COMPRISING 1-(4-CHLOROANILINO)-4-(4-PYRIDYLMETHYL)PHTHALAZINE AND A PH MODIFIER

(76) Inventors: Dieter Becker, Freiburg (DE); Nicoletta Loggia, Basel (CH); Barbara Lückel, Lorrach (DE); Andrea Kramer, Reute (DE); Stefanie Siepe, Freiburg (DE); Angelika Ries, Nenzlingen (CH)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/063,046

(22) PCT Filed: Aug. 21, 2006
(86) PCT No.: PCT/EP06/08216

§ 371 Date: Feb. 6, 2008

(30) Foreign Application Priority Data
Aug. 22, 2005 (GB) 0517205.1

(51) Int. Cl. A61K 31/502; A61P 9/00

(52) U.S. Cl. 514/248

(57) ABSTRACT

The present invention concerns pharmaceutical compositions comprising the pH dependent drug compound 1-(4-chloroanilino)-4-(4-pyridylmethyl)phthalazine and a pH modifier.

The present invention relates to solid pharmaceutical compositions comprising a drug compound with pH-dependent solubility, more particularly to pharmaceutical compositions comprising 1-(4-chloroanilino)-4-(4-pyridylmethyl)phthalazine or a pharmaceutically-acceptable salt thereof, preferentially the succinate salt, (hereinafter referred to as the “Agent”).

The Agent (drug) is well known from the literature; its structure and preparation being described for instance in WO 98/35958 or U.S. Pat. No. 6,258,812, which are hereby incorporated into the present application by reference. The Agent 1-(4-chloroanilino)-4-(4-pyridylmethyl)phthalazine succinate is also known as “PTK” or “PTK787” or “PTK/ZK” or “PTK787/ZK222584”.

The Agent is a potent orally active VEGF receptor tyrosine kinase inhibitor, which inhibits the vascular endothelial growth factor (VEGF) signal transduction by binding directly to the ATP-binding sites of VEGF receptors. The Agent reduces the microvasculature and inhibit growth of primary tumors and metastases and is useful for treating diseases associated with deregulated angiogenesis, especially neoplastic diseases (solid tumors), such as breast cancer, cancer of the colon, lung cancer, especially small cell lung cancer, and cancer of the prostate.

The Agent is a weakly basic drug compound that exhibits a significant pH dependent solubility along the gastrointestinal tract. The Agent is well soluble at low pH (pH 1; 80 g/L), e.g in the acidic environment of the fasted stomach, but significantly less soluble at higher physiological pH (pH 7; 7.1*10⁻⁴ g/L), e.g. at the site of absorption in the small intestine. As a result the Agent is prone to precipitate from solution as it passes from the acidic environment of the stomach to the higher pH environment of the upper gastrointestinal tract such as the small intestine. However, since the Agent's permeability is good in the small intestine, dissolution is the rate-limiting step to absorption in this part of the gastrointestinal tract. The pH of the gastrointestinal tract can also vary as a result, for example, whether a patient is in fed or fasted state, the use of certain medication, or certain medical conditions. Therefore, oral administration of such drug can result in a high inter- and intra subject variability.

Various concepts of improving the drug release of pharmaceutical compositions containing a drug with pH dependent solubility have been discussed, however there is a need for pharmaceutical composition comprising the Agent resulting in reduced inter- and intra subject variability and increased bioavailability.

Surprisingly, present inventors have identified improved pharmaceutical compositions comprising the Agent and a pH modifier. Shifting the microenvironmental pH to more acidic conditions inside the pharmaceutical composition results in an enhancement of the drug solubility and drug dissolution at pH conditions where the Agent exhibits reduced solubility. In addition, inter- and intra subject variability can be decreased. The extent and duration of pH modification depends on the physicochemical properties of the incorporated pH modifier and the polymer used.

In one aspect the present invention provides a pharmaceutical composition comprising:
(i) the Agent;
(ii) a pH modifier.

In a further aspect the present invention provides a pharmaceutical composition comprising:
(i) the Agent;
(ii) a pH modifier;
(iii) a polymer.

In a further aspect the present invention provides the use of Agent and excipients (pharmaceutical composition) for the preparation of a medicament for the treatment of patients with disorders associated with deregulated angiogenesis.

In a further aspect the present invention provides a method of orally administering Agent, e.g., for the treatment of disorders associated with deregulated angiogenesis, said method comprising orally administering to a patient in need of Agent therapy a pharmaceutical composition according to the present invention, preferentially administered once-a-day.

These and other features, advantages and objects of the present invention will be further understood and appreciated by those skilled in the art by references to the following specification, claims and appended drawings.

### BRIEF DESCRIPTION OF THE DRAWINGS

The accompanying drawings, which are incorporated in and constitute a part of the specification, illustrate exemplary embodiments of the present invention.

FIG. 1 shows the impact of fumaric acid on drug release from matrix tablets—in vitro using formulations according to Examples 3, 4 and 5.

FIG. 2 shows the impact of fumaric acid on drug release from matrix minitablets—in vitro using formulations according to Examples 3 and 5.

FIG. 3 shows the impact of fumaric acid on drug release—in vivo

A=Matrix tablet without fumaric acid according to Example 5;

A-FA=Matrix tablet with fumaric acid according to Example 3;

B=Matrix minitablets without fumaric acid according to Example 5;

B-FA=Matrix minitablets with fumaric acid according to Example 3.

FIG. 4 shows the reduction of variability in AUC₍₀₋₂₄ₕ₎ by incorporation of fumaric acid

A=Matrix tablet without fumaric acid according to Example 5;

A-FA=Matrix tablet with fumaric acid according to Example 3;

B=Matrix minitablets without fumaric acid according to Example 5;

B-FA=Matrix minitablets with fumaric acid according to Example 3.

### DETAILED DESCRIPTION OF INVENTION

As used herein the term “pH modifier” refers to an organic or inorganic chemical material that is able to release hydrogen ions (acid) like e.g. an organic or inorganic acid or an acidic polymer, e.g. Carbomers, or a latent acid, and is pharmaceutically acceptable. Latent acids are compounds that hydrolyze to a free acid in presence of water, e.g., glucono-δ-lactone.

In particular, the pH modifier may contain an acidic group having a pKa of 1 to 7, preferable of 2 to 6.5 or more preferable of 2.5 to 5.5. Where pKa values are mentioned herein, they are generally taken to be those as determined at a temperature of 25° C. in water.

For use in a sustained release pharmaceutical composition, pH modifier with a relative poor water solubility are preferred e.g. having a solubility of less than 5% (g/100 ml water) depending on the intended duration of action which is typically 1 to 24 hours, preferably 3 to 16 hours.

The use of solid acids or pharmaceutical acceptable salts thereof as pH modifier is particularly convenient for the manufacture of compositions according to the invention, which compositions are in the form of a solid dosage form.

In one preferred embodiment of the invention the pH modifier is an organic acid or a pharmaceutical acceptable salt thereof. Suitable organic acids contain one or more acidic groups, particularly compounds containing acidic groups selected from carboxylic and sulfonic acid groups, particularly those which are solid at ambient temperature and have 2 or more acidic groups. In addition functional groups that amplify or diminish the acidity of the acidic functional group can be present in the molecule like hydroxyl-groups or amino-groups

Particular water-soluble organic acids include a water-or poorly water soluble organic acid selected from a mono, di- or polybasic carboxylic acid and a mono, di or tri-sulfonic acid, preferably those which are solid at ambient temperature. Particular solid water-soluble carboxylic acids include, for example aliphatic mono or poly-carboxylic acids such as those containing from 1 to 20 carbon atoms, particularly from 2 to 6 carbon atoms, more particularly di- or tricarboxylic acids containing from 4 to 6 and especially 4 carbon atoms, any of which acids may be saturated or unsaturated or having branched or non-branched carbon atom chains. Examples of suitable solid water-soluble aliphatic mono-carboxylic acids include sorbic acid (2,4-hexandienoic acid). Examples of suitable solid water-soluble aliphatic di-carboxylic acids include adipic, malonic, succinic, glutaric, maleic or fumaric acid. The aliphatic carboxylic acid may be optionally substituted by one or more groups (for example 1, 2 or 3), which may be the same or different, selected from e.g. carboxy, amino or hydroxy. Suitable substituted solid water-soluble aliphatic carboxylic acids include for example hydroxy substituted aliphatic mono-carboxylic acids such as gluconic acid, solid forms of lactic acid, glycolic acid or ascorbic acid; hydroxy substituted aliphatic di-carboxylic acids such as malic, tartaric, tartronic (hydroxymalonic), or mucic (galactaric) acid; hydroxy 2 substituted aliphatic tri-carboxylic acids, for example citric acid; or amino acids carrying an acidic side chain, such as glutamic acid or aspartic acid.

Suitable aromatic carboxylic acids include water-soluble aryl carboxylic acids containing up to 20 carbon atoms. Suitable aryl carboxylic adds comprise an aryl group, for example a phenyl or naphthyl group which carries one or more carboxyl groups (for example 1, 2 or 3 carboxy groups). The aryl group is optionally substituted by one or more groups (for example 1, 2 or 3), which may be the same or different selected from hydroxy, (1-4C) alkoxy (for example methoxy) and sulfonyl. Suitable examples of aryl carboxylic acids include, for example benzoic, phthalic, isophthalic, terephthalic or trimellitic acid (1,2,4-benzene-tricarboxylic acid).

In another embodiment of the invention the pH modifier is a polymeric organic acid or a pharmaceutical acceptable salt thereof. The backbone of the polymer could be linear or branched or a mixture thereof. The backbone or the branches of the polymer could be in addition cross-linked by a suitable linker. Suitable polymeric acids contain a linear backbone with acidic groups, or a branched backbone with acidic groups or mixtures thereof. Suitable polymeric acids are e.g. synthetic high-molecular-weight polymers of acrylic acid that are crosslinked (e.g. Carbopol 71G) or methacrylic acid polymer crosslinked e.g. with divinylbenzene (e.g. Amberlite IRP-64). A further suitable polymeric acid is alginic acid.

Preferentially the pH modifier is selected from an organic acid, an acidic polymer, and a latent acid.

Even more preferred the pH modifier is selected from citric acid, fumaric acid, succinic acid, succinic acid anhydride, adipic acid and maleic acid or a pharmaceutical acceptable salt thereof including mixtures of two or more acids and/or salts.

Most preferred is fumaric acid as pH modifier.

Also most preferred is succinic acid or succinic acid anhydride as pH modifier.

Especially preferred is fumaric acid. Fumaric acid has a pKa of about 3, more particularly of 3.03, at 25° C.

In addition functional groups located on the polymer-backbone or in branches that amplify or diminish the acidity of the acidic functional group can be present like hydroxyl-groups or amino-groups.

In a preferred embodiment of the present invention the weight/weight ratio of pH-modifier to the acidic drug compound in the pharmaceutical composition is 0.005:1 or larger, preferably between 0.01:1 and 10:1, more preferred between 0.025:1 and 2:1, even more preferred between 0.5:1 and 2:1, most preferred about 1:1.

As used herein the term “polymer” refers to a polymer selected from the group that consists of cellulose derivatives [e.g., methyl cellulose, hydroxypropyl methyl cellulose, (e.g., hydroxypropyl methyl cellulose K100LV, K 4 M, hydroxypropyl methyl cellulose K 15 M), hydroxypropyl cellulose, hydroxyethyl cellulose, sodium-carboxy methyl cellulose, ethyl cellulose (e.g., ethyl cellulose 100), cellulose acetate (e.g., cellulose acetate CA-398-10 NF), cellulose acetate phthalate, cellulose acetate propionate, cellulose acetate butyrate, cellulose butyrate, cellulose nitrate, hydroxypropyl methyl cellulose phthalate, hydroxypropyl methyl cellulose acetate succinate]; acryl derivatives [e.g., polyacrylates, cross-linked polyacrylates], methycrylic acid copolymers, vinyl polymers (e.g., polyvinyl pyrrolidones, polyvinyl acetates, polyvinyl acetate phthalates) and its mixtures, as marketed under the trade name Kollidon SR®, polyethylene glycols, polyanhydrides, polysaccharides (e.g., xanthans, xanthan gum), galactomannan, pectin, and alginates. The polymer may also serve in addition the function of a pH modifier.

A preferred polymer is hydroxypropyl methyl cellulose.

In certain exemplary embodiments of the present invention, the pharmaceutical composition may comprise additional excipients commonly found in pharmaceutical compositions, examples of such excipients include, but are not limited to, fillers, glidants, lubricants, binders, antioxidants, antimicrobial agents, enzyme inhibitors, stabilizers, preservatives, flavors, sweeteners and other components as described in Handbook of Pharmaceutical Excipients, Rowe et al., Eds., 4^{th} Edition, Pharmaceutical Press (2003), which is hereby incorporated by reference.

Additional excipients with the exception of fillers and/or binders may comprise from about 0.05-11% by weight of the total pharmaceutical composition, e.g. from about 0.5 to about 3.5% by weight of the total composition. Antioxidants, anti-microbial agents, enzyme inhibitors, stabilizers or preservatives typically provide up to about 0.05-1% by weight of the total pharmaceutical composition. Sweetening or flavoring agents typically provide up to about 2.5% or 5% by weight of the total pharmaceutical composition. Lubricants typically provide up to about 0.5% to 3%, preferentially about 1%, by weight of the total pharmaceutical composition.

Examples of a “lubricant”, as used herein, include, but are not limited to magnesium stearate, talc, hydrogenated castor oil, glycerylbehaptate, glycerolmonostearate, polyethylene glycol, ethylene oxide polymers, sodium lauryl sulfate, magnesium lauryl sulfate, sodium oleate, sodium stearyl fumarate, DL-leucine, collidal silica, and others known in the art.

Examples of a “filler”, as used herein, include, but are not limited to lactose, (which may be in an anhydrous or hydrated form), sugar, starches (for example corn, wheat, maize, potato), modified starches (e.g., starch hydrolysates or pregelatinized starch), mannitol, sorbitol, trehalose, maltose, glucose anhydrate; inorganic salts (e.g., calcium carbonate, magnesium carbonate, dibasic calcium phosphate, tribasic phosphate, calcium sulfate), microcrystalline cellulose, cellulose derivates.

Examples of a “glidant” as used herein, include, but are not limited to Aerosil 200 or talc.

Examples of a “binder” as used herein, include, but are not limited to hydroxypropylmethyl-cellulose (HPMC), e.g. HMPC with a low apparent viscosity, e.g. below 100 cps as measured at 20° C. for a 2% by weight aqueous solution, e.g. below 50 cps, preferably below 20 cps, for example HPMC 3 cps, as known and commercially available under the name Pharmacoat® 603 from the Shin-Etsu company, other suitable binders for a composition of the present are polyvinylpyrrolidone (PVP), e.g. PVP K30 or PVP K12, as known and commercially available under the trade name Povidone® from the BASF company;

Examples of antioxidants include, but are not limited to, ascorbic acid and its derivatives, tocopherol and its derivatives, butyl hydroxyl anisole and butyl hydroxyl toluene. Vitamin E as α-tocopherol is particularly useful.

The dosage forms of this invention can be widely implemented. For purposes of discussion, not limitation, the many embodiments hereunder can be grouped into three classes according to design and principle of operation.

1. The first class of dosage forms described below include but are not limited to modified release hydrophilic swelling, eroding, dispersible or dissolvable monolithic matrix tablets or compression-coated matrix tablets containing all or partial amounts of the acid in the core tablet or multiparticulate matrix systems such as minitablets, granules, or pellets.
2. The second class of dosage forms consists of coated modified release multiparticulates systems where release of the drug is generally modulated by a membrane, such as coated minitablets, pellets, granules or beads including those using crystals of the acid as starter cores. Compared to monolithic systems, multiparticulates display the advantage that the mean gastric emptying is faster and less dependent on the nutritional state as they are sufficiently small to be evacuated through the pylorus. Multiparticulates can have numerous formulation applications. For example, they may be filled in a capsule shell or as a sachet or they may be compressed into a tablet. When the composition is in the form of a tablet, it is preferably a tablet which is able to disintegrate or dissolve in the mouth, stomach or small intestine to give modified release coated multiparticles.
3. The third class of dosage forms consists of mixtures of two or more multiparticulates e.g. immediate release (IR) and modified release (MR) or multiparticulates having 2 or more different modified release profiles, which may be filled in a capsule shell or as a sachet or compressed into a tablet. The overall release of drug from such system on administration of the dosage form will then be characterized by the ratio of the different single release units and their specific drug release profile.
4. The fourth class of dosage forms consists of bilayer-tablets consisting of an IR and an MR layer or of 2 MR layers of different release profiles. In a further embodiment, also trilayer tablets made from two outer MR layers and an inner layer of pure acid or pure acid and filler are comprised.

In a further embodiment of the present invention, one or more of the single specific release units dosage forms disclosed in this invention are additionally coated with an enteric polymer which prevents drug dissolution from the solid dosage form before reaching the small intestine

In a further embodiment of the present invention, a subcoat is applied separating the enteric coating from the pH modifier comprising matrix.

Enteric coating (% of final weight) contains for instance
2-40% polymers for enteric coating (e.g. Hydroxypropylmethylcellulose phthalate (i.e. HP 50, HP 55 from Shin Etsu), Hydroxypropylmethylcellulose acetate succinate (i.e. Aqoat types H, M, L from Shin Etsu), Methyl acrylic acid-ethyl acylic acid copolymer (Methacrylic acid copolymer, USP) (i.e. Eudragit L, S, L100-55, L30D from Röhm Pharma, Acryl-Eze from Colorcon, Kollicoat MAE 30 DP from BASF), Celluloseacetatephthalate, (i.e. Aquacoat CPD from FMC Biopolymer, or Polymer from Eastman Kodak) Polyvinylacetatephthalate (Sureteric, Colorcon)
0-15% polymers for subcoating (isolation coat between tablet core and enteric coat): Hydroxypropylmethylcellulose (Pharmacoat 603 or 606), ethylcellulose (i.e. Aquacoat ECD, FMC Biopolymer, Surelease, Colorcon) and or mixtures thereof with a ratio of Ethylcellulose:HPMC=1:1 up to 1:10), Polyvinylalcohole (Opadry II HP, type 85F, Colorcon)
0-10% plastisizers (triacetine, triethylcitrate, PEG 4000, PEG 6000, PEG 8000, Diethylphthalate, Diethylsebacate, Acetyltriethylcitrate etc.)
0-15% antisticking agents (Aerosil 200, Syloid 244 FP, Talcum, Glycerol monostearate etc.)
organic solvents or mixtures thereof with and without parts of water (ethanol, acetone, isopropanol) or water q.s. to dissolve or disperse the coating polymers and excipients for coating solution
0-0.5% sodium hydroxide for redispersion of polymers for aqueous enteric coating suspensions (i.e. Eudragit L100-55)

The following examples are illustrative, but do not serve to limit the scope of the invention described herein. The examples are meant only to suggest a method of practicing the present invention. Quantities of ingredients, represented by percentage by weight of the pharmaceutical composition, used in each example are set forth in the respective tables located after the respective descriptions.

### EXAMPLES

### 1. Modified Release Hydrophilic Swelling, Eroding, Dispersible or Dissolvable Monolithic Matrix Tablets or Multiparticulate Systems Such as Minitablets, Pellets or Granules

### 1.1 Formulation Ingredients and Ranges

1-80% Agent
1-60% pH modifier (e.g. citric acid, fumaric acid, succinic acid, adipic acid, maleic acid)
10-60% water-soluble and water-insoluble polymers (e.g. Methocel K100M, Methocel K4M, Methocel K100LV or mixtures thereof; Kollidon SR)
0-2% Aerosil 200
0-2% Magnesium stearate
optionally additional tabletting excipients, e.g., fillers (3-65%, preferable 4-55%) such as lactose and binders (0.5-5%, preferable 2-3%) such as HPMC 3 cps

### 1.2 Preparation of Granules for Tablets Including Minitablets and Compression-Coated Tablets

The active ingredient, the pH modifier, the polymer, and any additional tabletting excipients are mixed and wet granulated by water or organic solvents. The dried granules are e.g. either

1) sieved through an 800 μm sieve and filled in a capsule or sachet, or
2) sieved through an 800 μm sieve and compressed in a monolithic matrix tablets including compression-coated tablets or
3) sieved through a 400 μm sieve and compressed in minitablets.

For compression purposes, an outer phase consisting of Aerosil and magnesium stearate is added and mixed thoroughly. The blend is compressed into monolithic matrix tablets of, e.g., a diameter of 5 to 12 mm or minitablets of a diameter of e.g. 1.7 to 2 mm.

### 1.3 Preparation of Pellets

In a further embodiment, the polymer, the pH modifier and any adjuvant (preferably cellulose, cellulose derivatives, and lactose) are processed into pellets by means of extrusion and subsequent spheronization.

Another subject of the invention is a process for the production of pellets by means of direct pelletization. In this case, the starting substances are mixed and processed into pellets by means of a binder solution (wet granulation) or melted additives (e.g., fats).

Another subject of the invention is a process for the production of pellets by means of spray-drying or spray-solidification.

Another subject of the invention is a process for the production of pellets by means of rotor granulation.

1.4 Composition of Matrix Tablets and Matrix Minitablets (Either with or without Enteric Coat) Including the Outer Layer of Compression-Coated Tablets

Tablets were prepared with a weight of 250±5 mg (Ø10 mm). 250 mg±5 mg of the prepared minitablets (1-2 mm) were filled into capsules:

Example 1

| | | mg/tablet/ |
|---|---|---|
| | [%] | capsule |
| Methocel K100LV | 30.00 | 75.00 |
| PTK787 | 10.00 | 25.00 |
| Fumaric acid | 20.00 | 50.00 |
| Lactose milled | 34.80 | 87.00 |
| HPMC 3 cps | 2.67 | 6.68 |
| Mg-Stearat | 1.00 | 2.50 |
| Aerosil | 1.53 | 3.83 |
| | 100.00 | 250.00 |

Example 2

| | | mg/tablet/ |
|---|---|---|
| | [%] | capsule |
| Methocel K100LV | 30.00 | 75.00 |
| PTK787 | 10.00 | 25.00 |
| Fumaric acid | 0.00 | 0.00 |
| Lactose milled | 54.80 | 137.00 |
| HPMC 3 cps | 2.67 | 6.68 |
| Mg-Stearat | 1.00 | 2.50 |
| Aerosil | 1.53 | 3.83 |
| | 100.00 | 250.00 |

Example 3

| | | mg/tablet/ |
|---|---|---|
| | [%] | capsule |
| Methocel K100LV | 30.00 | 75.00 |
| PTK787 | 20.00 | 50.00 |
| Fumaric acid | 20.00 | 50.00 |
| Lactose milled | 24.80 | 62.00 |
| HPMC 3 cps | 2.67 | 6.68 |
| Mg-Stearat | 1.00 | 2.50 |
| Aerosil | 1.53 | 3.83 |
| | 100.00 | 250.00 |

Example 4

| | | mg/tablet/ |
|---|---|---|
| | [%] | capsule |
| Methocel K100LV | 30.00 | 75.00 |
| PTK787 | 20.00 | 50.00 |
| Fumaric acid | 40.00 | 100.00 |
| Lactose milled | 4.80 | 12.00 |
| HPMC 3 cps | 2.67 | 6.68 |
| Mg-Stearat | 1.00 | 2.50 |
| Aerosil | 1.53 | 3.83 |
| | 100.00 | 250.00 |

EXAMPLE 5

| | | mg/tablet/ |
|---|---|---|
| | [%] | capsule |
| Methocel K100LV | 30.00 | 75.00 |
| PTK787 | 20.00 | 50.00 |
| Fumaric acid | 0.00 | 0.00 |
| Lactose milled | 44.80 | 112.00 |
| HPMC 3 cps | 2.67 | 6.68 |
| Mg-Stearat | 1.00 | 2.50 |
| Aerosil | 1.53 | 3.83 |
| | 100.00 | 250.00 |

EXAMPLE 6

| | | mg/tablet/ |
|---|---|---|
| | [%] | capsule |
| Methocel K4M | 30.00 | 75.00 |
| PTK787 | 10.00 | 25.00 |
| Fumaric acid | 20.00 | 50.00 |
| Lactose milled | 34.00 | 85.00 |
| HPMC 3 cps | 2.67 | 6.68 |
| Mg-Stearat | 1.33 | 3.33 |
| Aerosil | 2.00 | 5.00 |
| | 100.00 | 250.00 |

EXAMPLE 7

| | | mg/tablet/ |
|---|---|---|
| | [%] | capsule |
| Methocel K4M | 30.00 | 75.00 |
| PTK787 | 10.00 | 25.00 |
| Fumaric acid | 0.00 | 0.00 |
| Lactose milled | 54.00 | 135.00 |
| HPMC 3 cps | 2.67 | 6.68 |
| Mg-Stearat | 1.33 | 3.33 |
| Aerosil | 2.00 | 5.00 |
| | 100.00 | 250.00 |

EXAMPLE 8

| | | mg/tablet/ |
|---|---|---|
| | [%] | capsule |
| Methocel K100LV | 30.00 | 75.00 |
| Lactose milled | 34.00 | 85.00 |
| PTK787 | 30.00 | 75.00 |
| Fumaric acid | 0.00 | 0.00 |
| HPMC 3 cps | 2.67 | 6.68 |
| Mg-Stearat | 1.33 | 3.33 |
| Aerosil | 2.00 | 5.00 |
| | 100.00 | 250.00 |

EXAMPLE 9

| | | mg/tablet/ |
|---|---|---|
| | [%] | capsule |
| Methocel K100LV | 30.00 | 75.00 |
| PTK787 | 30.00 | 75.00 |
| Fumaric acid | 15.00 | 37.50 |
| Lactose milled | 19.00 | 47.50 |
| HPMC 3 cps | 2.67 | 6.68 |
| Mg-Stearat | 1.33 | 3.33 |
| Aerosil | 2.00 | 5.00 |
| | 100.00 | 250.00 |

EXAMPLE 10

| | | mg/tablet/ |
|---|---|---|
| | [%] | capsule |
| Methocel K100LV | 30.00 | 75.00 |
| Lactose milled | 4.00 | 10.00 |
| PTK787 | 30.00 | 75.00 |
| Fumaric acid | 30.00 | 75.00 |
| HPMC 3 cps | 2.67 | 6.68 |
| Mg-Stearat | 1.33 | 3.33 |
| Aerosil | 2.00 | 5.00 |
| | 100.00 | 250.00 |

EXAMPLE 11
Composition of granules

| | [%] | mg/capsule |
|---|---|---|
| Methocel K100LV | 30.00 | 75.00 |
| Lactose grounded | 27.33 | 68.33 |
| Fumaric acid | 20.00 | 50.00 |
| PTK787 | 20.00 | 50.00 |
| HPMC 3 cps | 2.67 | 6.68 |

EXAMPLE 12
Composition of pellets

| | [%] | mg/capsule |
|---|---|---|
| Methocel K100LV | 30.00 | 75.00 |
| Avicel PH101 | 27.33 | 68.33 |
| Fumaric acid | 20.00 | 50.00 |
| PTK787 | 20.00 | 50.00 |
| HPMC 3 cps | 2.67 | 6.68 |

### Compression-Coated Tablets Comprising the Acid Completely in the Inner Tablet Core or Partially in the Core and Partially in the Outer Layer

For the preparation of the inner core tablet comprising the acid, tablets were made from pure acid mixed with a lubricant, e.g. from pure succinic acid, fed manually into the die of a single-punch tabletting machine (EK0, Korsch, Germany). In another embodiment, the inner core tablet was compressed from granules made of acid and a filler, water-soluble or water-insoluble, preferably water-insoluble, mixed with a lubricant. The matrix granules for the outer layer were prepared according to the method described above (1.2. and 1.4), but could also comprise only drug and polymer without any acid.

For the compression-coated tablet, the core tablet was placed in the center of the outer layer (e.g. the granules of the outer layer were filled into the die to make a powder bed, on the center of which the core tablet was placed before being covered by further granules of the outer layer) and a compression force was being applied.

EXAMPLE 13
Composition of a compression-coated tablet made from matrix granules
comprising succinic acid as outer layer and a succinic acid comprising core

| | Weight | | % | % |
|---|---|---|---|---|
| Outer layer | | | | |
| PTK787 (125 mg) | 167.5 | mg | 31.3 | 21.3 |
| Methocel K100LV | 250.0 | mg | 46.7 | 31.8 |
| lactose monohydrate | 20.0 | mg | 3.7 | 2.5 |
| succinic acid | 85 mg | mg | 15.9 | 10.8 |
| magnesium stearate | 7.5 | mg | 1.4 | 1.0 |
| Aerosil | 5.0 | mg | 0.9 | 0.6 |
| | 535.0 | mg | 100.0 | |
| Inner core | | | | |
| succinic acid | 250.0 | mg | | 31.8 |
| Total | 785.0 | mg | | 100.0 |

### 1.5 Preparation of Enteric Coating and Subcoating

The isolation coat is applied from an aqueous solution of HPMC (4-8%), plactisizer (0-3%) and antisticking agents (0-3%). Aquacoat ECD or Surelease (aqueous ethylcellulose dispersion) might be added in the range of 1:10 up to 1:1 (ethylcellulose:HPMC) to improve the isolation effect of the subcoating. Based on the tablet size the total amount of subcoat applied is 2-15% (more preferred: large tablets 4-10%, minitablets/pellets: 8-15%). Polyvinylalcohol (Opadry II HP) in a range of 2-10% of core weight can be employed for an effective subcoating.

Furthermore, a HPMC subcoat could be coated from organic suspension in ethanol/acetone 1:1 (about 6-10% polymer per solvent) without any further additives.

In case of an organic enteric coating solution, after dissolving the enteric coating polymer and the plastisizer in organic solvents, the antisticking agents are dispersed. With regard to coating from aqueous dispersions, the plastisizer is dissolved or finely dispersed in water, the antisticking agent is dispersed, and finally the reconstituted suspension (i.e., Aqoat or Eudragit L 100-55) or the commercially available aqueous polymer dispersion (Eudragit L 30D, Acryl-Aze, Kollicoat MAE 30 D) are added.

The coating is applied using a pan coater or fluidized bed coater with or without Wurster principle up to a coating layer between 2 and 45% (more preferred about 10-25% for large tablets and 20-40% for small tablets/minitablets) at a product temperature between 28 and 50° C. Subcoating layer 2-15% (more preferred: large tablets 4-10%, minitablets/pellets: 8-15%)/enteric coating layer: 5-40% (more preferred: large tablets: 8-20%, minitablets/pellets: 15-30%). The layer depends on the tablet size to assure an enteric resistance for 1-3 hours in artificial gastric juice or 0.1 n HCL solution (acc. to Ph Eur. or USP). Additionally, swelling of the tablet core during gastric resistance test should be reduced to a minimum.

EXAMPLE COATING A

| | parts | % | mg/250 mg core | mg/8 mg core |
|---|---|---|---|---|
| Subcoat | | | | |
| HPMC 3 cps | 5 | 25 | 12.5 | 0.8 |
| Trietylcitrate | 0.5 | 2.5 | 1.25 | 0.08 |
| Talc | 0.5 | 2.5 | 1.25 | 0.08 |
| Water q.s. | | | | |
| Enteric coat | | | | |
| Eudragit L 30 D (dry) | 10 | 50 | 25 | 1.6 |
| PEG 6000 | 2 | 10 | 5 | 0.32 |
| Syloid 244 FP | 2 | 10 | 5 | 0.32 |
| Water q.s | | | | |
| Total (dry) | 20 | 100 | 50 | 3.2 |

EXAMPLE COATING B

| | parts | % | mg/250 mg core | mg/8 mg core |
|---|---|---|---|---|
| Subcoat | | | | |
| HPMC 3 cps | 6 | 26.67 | 15.0 | 0.96 |
| Aquacoat ECD (dry) | 2 | 8.89 | 5.0 | 0.32 |
| Trietylcitrate | 0.6 | 2.67 | 1.5 | 0.096 |
| Glycerol | 0.4 | 1.77 | 1.0 | 0.064 |
| monostearate | | | | |
| Water q.s. | | | | |
| Enteric coat | | | | |
| HPMC AS (Aqoat) | 10 | 44.44 | 25.0 | 1.60 |
| MF | | | | |
| Triethylcitrate | 2.5 | 11.11 | 6.25 | 0.40 |
| Talc | 1 | 4.44 | 2.5 | 0.16 |
| Water q.s. | | | | |
| Total (dry) | 22.5 | 100.0 | 56.25 | 3.60 |

EXAMPLE COATING C

| | parts | % | mg/250 mg core | mg/8 mg core |
|---|---|---|---|---|
| Subcoat | | | | |
| HPMC 3 cps | 5 | 32.5 | 12.5 | 0.8 |
| Ethanol/Acetone | | | | |
| 1.1: q.s. | | | | |
| Enteric coat | | | | |
| HP 50 | 8 | 51.9 | 20.0 | 1.28 |
| Triacetine | 0.8 | 5.2 | 2.0 | 0.13 |
| Aerosil 200 | 1.6 | 10.4 | 4.0 | 0.26 |
| Ethanol/Acetone | | | | |
| 1:1: q.s. | | | | |
| Total (dry) | 15.4 | 100.0 | 38.5 | 2.47 |

EXAMPLE COATING D

| | parts | % | mg/250 mg core | mg/8 mg core |
|---|---|---|---|---|
| Subcoat | | | | |
| Opadry II HP | 4 | 21.46 | 10 | 0.64 |
| Water q.s. | | | | |
| Enteric coat | | | | |
| Eudragit L100-55 | 10 | 53.65 | 25 | 1.6 |
| Sodium hydroxide | 0.14 | 0.75 | 0.35 | 0.022 |
| Triethylcitrate | 2.5 | 13.41 | 6.25 | 0.4 |
| Syloid 244 FP | 2 | 10.73 | 5.0 | 0.32 |
| Water q.s | | | | |
| Total dry | 18.64 | 100.0 | 46.6 | 2.982 |

EXAMPLE COATING E

| | parts | % | mg/250 mg core | mg/8 mg core |
|---|---|---|---|---|
| Enteric coat | | | | |
| HP 50 | 10 | 71.43 | 25 | 1.6 |
| Diethylsebacate | 1 | 7.14 | 2.5 | 0.16 |
| Talc | 3 | 21.43 | 7.5 | 0.48 |
| Ethanol/Acetone | | | | |
| 1:1 q.s. | | | | |
| Total dry | 14 | 100.0 | 34 | 2.24 |

EXAMPLE COATING F

| | parts | % | mg/250 mg core | mg/8 mg core |
|---|---|---|---|---|
| Enteric coat | | | | |
| Eudragit L 100-55 | 10 | 76.92 | 25 | 1.6 |
| Triethylcitrate | 1 | 7.69 | 2.5 | 0.16 |
| Syloid 244 FP | 2 | 15.38 | 5.0 | 0.32 |
| Isopropanol/Water | | | | |
| 97.3: q.s | | | | |
| Total dry | 13 | 100.0 | 32.5 | 2.08 |

### 2. Modified release coated multiparticulates systems such as minitablets, pellets, Granules or Beads

### 2.1 Preparation of Multiparticulate Systems

### 2.1.1 Preparation of Granules and Minitablets

The active ingredient, the pH modifier, and any additional tabletting excipients are mixed and wet granulated by water or organic solvents. The dried granules are e.g., either sieved through an 800 μm for the granule preparation or sieved through a 400 μm sieve and compressed in minitablets. For compression purposes, an outer phase consisting out of Aerosil and magnesium stearate was added and mixed thoroughly. The blend is compressed into minitablets of a diameter of e.g. 1.7 to 2 mm. The resulting granules and minitablets are finally coated with one of a coating formulations using polymers as described below (i.e. diffusion coat, diffusion coat with an additional enteric coat, diffusion coat comprising an enteric polymer).

EXAMPLE 14
Composition of minitablets

| | | mg/250 mg |
|---|---|---|
| | % | tablets |
| Lactose anhydrous | 24.14 | 60.35 |
| Fumaric acid | 20.00 | 50.00 |
| PTK787 | 20.00 | 50.00 |
| Avicel PH 102 | 33.33 | 83.33 |
| Aerosil 200 | 1.53 | 3.83 |
| Mg-stearat | 1.00 | 2.50 |
| | 100.00 | 250.00 |

EXAMPLE 15
Composition of granules

| | [%] | mg/capsule |
|---|---|---|
| Avicel PH102 | 30.00 | 75.00 |
| Lactose grounded | 27.33 | 68.33 |
| Fumaric acid | 20.00 | 50.00 |
| PTK787 | 20.00 | 50.00 |
| HPMC 3 cps | 2.67 | 6.68 |

### 2.1.2 Preparation of Pellets:

In one embodiment, a dry blend is made by mixing the drug, the pH modifier, micro-crystalline cellulose (i.e., Avicel PH101) and lactose in a planetary mixer. Purified water is added to give a wet mass that is subsequently extruded using a screen of a suitable size. The extrudates are rounded in a spheroniser, thoroughly dried and sieved for suitable size selection, obtaining immediate release pellets. Any other pellet forming process as mentioned under 1.3. may also be used. The resulting pellets are finally coated with one of a coating formulations as described below (i.e., diffusion coat, diffusion coat with an additional enteric coat, diffusion coat comprising an enteric polymer). Coated pellets can then be dispensed in a capsule or sachets.

Additionally, immediate and modified release pellets can be used as a combination by including them into the same capsule or sachets.

EXAMPLE 16
Composition of pellets (amounts given in %)

| | | | |
|---|---|---|---|
| PTK 787 | 20% | 30% | 10% |
| Fumaric acid | 20% | 20% | 20% |
| Lactose (standard quality) | 10% | 8% | 15% |
| Microcrystalline cellulose (Avicel PH101) | 50% | 42% | 55% |
| Water for wet massing | q.s.* | q.s.* | q.s*. |

*removed during processing.

### 2.1.3 Preparation of Beads Based on Soluble and Insoluble Non-Pareil Seeds as Well as Rounded pH Modifier Starter Cores as Well as Drug Particles

2.1.3.1 In one embodiment, drug solutions are prepared by dissolving the drug, the pH modifier and the remaining formulation components as described below in the selected media with mixing. Non-pareil seeds, i.e. drug-free cores, are dispensed into a Wurster fluid bed coater and fluidized. The drug solution previously prepared is then sprayed onto the seeds until the drug solution is depleted obtaining immediate release beads. The beads are dried in the same conditions for 5 minutes. The resulting beads are again dispensed into a Wurster fluid bed coater and finally coated with an aqueous dispersion or an organic solution of the coating ingredients of the coating formulation formulations below (diffusion coat, diffusion coat with an additional enteric coat, diffusion coat comprising an enteric polymer), obtaining the modified release beads.

Coated beads can then be dispensed in a capsule or sachets.

Additionally, immediate and modified release beads can be used as a combination by including them into the same capsule or sachet.

EXAMPLE 17
Composition of beads to be applied onto 1000 g non-pareil seeds (amounts
given in %)

| | [%] | [%] | [%] |
|---|---|---|---|
| PTK787 | 40 | 30 | 20 |
| Fumaric acid | 40 | 30 | 40 |
| Pharmacoat 615 | 18 | 36 | 36 |
| PEG 400 | 2 | 4 | 4 |
| Ethanol/Water 70/30 | | | |

Fumaric acid: 1-60%
PTK787: 20-70%
Pharmacoat: 10-50%

2.1.3.2 In a second embodiment, non-pareil seeds are dispensed into a Wurster fluid bed coater. After fluidisation, spraying of the drug layer solution as per formulation A is commenced to layer drug solution effectively onto the seeds. Spraying is continued until the drug layer solution is exhausted. A protective layer consisting of a solution of hydroxypropyl-methylcellulose (Opadry™ clear) in purified water may then be sprayed onto the seeds. Spraying is continued until the HPMC solution is exhausted. Then, a solution of an organic acid and HPMC as per formulation B is sprayed onto the seeds. The beads are dried, under the same conditions for 5 minutes, obtaining the immediate release beads. Additionally, a solution of hydroxypropylmethylcellulose (Opadry™) in purified water can be sprayed onto the seeds. The resulting beads are again dispensed into a Wurster fluid bed coater and finally coated with an aqueous dispersion or an organic solution of the coating ingredients of the coating formulation formulations below (diffusion coat, diffusion coat with an additional enteric coat, diffusion coat comprising an enteric polymer), obtaining the modified release beads. Finally, a solution of hydroxypropyl methylcellulose (Opadry™) in purified water may be sprayed onto the seeds. The beads are dried, under the same conditions for 5 minutes, obtaining the modified release beads.

Coated beads can then be dispensed in a capsule or sachets.

Additionally, immediate and modified release beads can be used as a combination by including them into the same capsule or sachet (compare 3.3).

EXAMPLE 18
Composition of beads to be applied onto 1000 g non pareil seeds
(amounts given in %)
Formulation A (amounts given in %)

| | | | |
|---|---|---|---|
| PTK787 | 80% | 60% | 40% |
| Hydroxypropyl methylcellulose(Methocel E50LV) | 18% | 36% | 54% |
| Polyethylene glycol (PEG 400) | 2% | 4% | 6% |
| Ethanol/Water (70:30) | q.s.* | q.s.* | q.s.* |

Formulation B (amounts given in %)

| | | | |
|---|---|---|---|
| Fumaric acid | 80% | 60% | 40% |
| Hydroxypropyl methylcellulose(Methocel E50LV) | 18% | 36% | 54% |
| Polyethylene glycol (PEG 400) | 2% | 4% | 6% |
| Ethanol/Water (70:30) | q.s.* | q.s.* | q.s.* |

*removed during processing

2.1.3.3 In a third embodiment, non-pareil seeds are dispensed into a Wurster fluid bed coater. After fluidisation, spraying of the solution comprising pH modifier and HPMC as per formulation B (see second embodiment) is commenced to layer drug solution effectively onto the seeds. Spraying is continued until the pH modifier layer solution is exhausted. A protective layer consisting of a solution of hydroxypropyl methylcellulose (Opadry™ clear) in purified water may then be sprayed onto the seeds. Spraying is continued until the HPMC solution is exhausted. Then, a solution of the drug as per formulation A (see second embodiment) is sprayed onto the seeds. Spraying is continued until the drug layer solution is exhausted. The beads are dried, under the same conditions for 5 minutes, obtaining the immediate release beads. Additionally, a solution of hydroxypropyl methylcellulose (Opadry™) in purified water can be sprayed onto the seeds. The resulting beads are again dispensed into a Wurster fluid bed coater and finally coated with an aqueous dispersion or an organic solution of the coating ingredients of the coating formulation formulations below (diffusion coat, diffusion coat with an additional enteric coat, diffusion coat comprising an enteric polymer), obtaining the modified release beads. Finally, a solution of hydroxypropyl methylcellulose (Opadry™) in purified water may be sprayed onto the seeds. The beads are dried, under the same conditions for 5 minutes, obtaining the modified release beads.

Coated beads can then be dispensed in a capsule or sachets.

Additionally, immediate and modified release beads can be used as a combination by including them into the same capsule or sachet.

2.1.3.4 In a fourth embodiment, rounded starter cores of the pH modifier with an average diameter of e.g. 0.3 to 1 mm are sprayed uniformly with an alcoholic polymer solution, e.g. comprising PVP, in a suitable vessel and are mixed with a mixture of the drug and the pH modifier until the beads roll freely again. After drying, this operation is operation is repeated until the desired total amount of the drug has been applied. However, it is also possible to dissolve or suspend the drug in the adhesive solution and to apply this solution or suspension uniformly onto the surface of the starter cores.

Suitable bonding agents include adhesive solutions such as starch paste, sugar syrup, and solution of gelatin, guar rubber, cellulose ether (e.g. HEC, HPMC), or PVP. The acid in the starter core can be different from the acids admixed with the drug. Especially suitable for the starter cores are those acids which have an approximately spherical shape, e.g. tartaric acid, citric acid, malic acid, succinic acid, ascorbic acid.

2.1.3.5 In a fifth embodiment, the invention also relates to a process whereby the polymer coating, the pH modifier and the adjuvant are processed into beads by the layered application onto the drug (layering).

### 2.2 Diffusion Coating Compositions

### 2.2.1 Coating Ingredients & Ranges

1-20% polymers for diffusion coating e.g. Ethylcellulose (Aquacoat ECD, FMC Biopolymer, Surrelease, Colorcon), Acrylic/methacrylic acid-ester/Eudragit RL, Eudragit RS (Röhm)
0-20% water soluble polymers as pore formers, i.e. Hydroxypropylmethylcellulose 3, 6 cps (Pharmacoat 603, 606, Shin-Etsu), Polyethylenglycole (PEG 2000-PEG 8000)
0-15% polymers for subcoating (isolation coat between tablet core and enteric coat): Hydroxypropylmethylcellulose (Pharmacoat 603 or 606), ethylcellulose (i.e. Aquacoat ECD, FMC Biopolymer, Surelease, Colorcon) and or mixtures thereof with a ratio of Ethylcellulose:HPMC=1:1 up to 1:10), Polyvinylalcohole (Opadry II HP, type 85F, Colorcon)
0-20% enteric coating polymers as pore formers (list of potential polymers, see above)
0-10% plastisizers (triacetine, triethylcitrate, PEG 4000, PEG 6000, PEG 8000, Diethylphthalate, Diethylsebacate, Dibuthylsebacate, Acetyltriethylcitrate etc.)
0-15% antisticking agents (Aerosil 200, Syloid 244 FP, Talcum, Glycerol monostearate etc.)
organic solvents or mixtures thereof with and without parts of water (ethanol, acetone, isopropanol) or water q.s. to dissolve or disperse the coating polymers and excipients for coating solution

### 2.2.2 Coatings Based on Acrylic/Methacrylic Acid Ester Polymers

The polymers used for diffusion coating are Eudragit RS/RL mixtures in a ratio of 1:1 up to 9:1 from aqueous suspension or organic solution. Suitable plastisizers are triethylcitrate, dibutylsebacate, Triacetine in a range of 1 to 30% of coating dispersion (5-20%). Eudragit RS could be combined with the enteric coating polymer as pore former like Hydroxypropylmethylcellulose acetate succinate, Type Aqoat type M(MF) or H (HF) in organic solution or aqueous dispersion or with Hydroxypropylmethylcellulose phthalate (i.e. HP 50, HP 55) in organic solution. An enteric pore former suppresses the drug release in the acidic environment in the stomach. After solution of the enteric pore former in intestinal juice with ph>5.5 the drug will dissolve and uniformly owing to the low microenvironmental pH inside the solid dosage form. Thereby, less inter- and intra subject variance is expected.

The coating layer is applied between 5 and 30%, most probably between 7 and 15%, i.e. 10% of core weight. The ratio of Eudragit RS and enteric pore former may be varied between 95:5 up to 50:50 to adapt the release profile.

EXAMPLE COATING G

| | parts | % (dry) | % (liquid) | mg/8 mg core |
|---|---|---|---|---|
| Eudragit RL 30 D | 1.52 | 6.9 | 4.62 | 0.06 |
| Eudragit RS 30D | 13.76 | 62 | 41.70 | 0.50 |
| Triethylcitrate | 2.8 | 12.5 | 2.80 | 0.10 |
| Syloid 244 FP | 4.18 | 18.6 | 4.18 | 0.15 |
| Water. | | q.s. | 46.70 | |
| Total | 22.26 | 100 | 100.00 | 0.80 |

Optional Ratio: Eudragit RS:RL 9:1
Coating layer: 5-20% (i.e. 10%) of core weight

EXAMPLE COATING H

| | parts (dry) | % (dry) | % (liquid) | mg/8 mg core |
|---|---|---|---|---|
| Eudragit RL 12.5 | 1.28 | 17.75 | 30.93 | 0.14 |
| Eudragit RS 12.5 | 3.86 | 53.54 | 10.30 | 0.43 |
| Triethylcitrate | 0.52 | 7.21 | 0.52 | 0.06 |
| Syloid 244 FP | 1.55 | 21.50 | 1.55 | 0.17 |
| Acetone | q.s. | q.s. | 28.35 | |
| Isopropanol | q.s. | q.s. | 28.35 | |
| Total | 7.21 | 100.00 | 100.00 | 0.80 |

Optional Ratio: Eudragit RS:RL 7.5:2.5
Coating layer: 5-20% (i.e. 10%) of core weight

EXAMPLE COATING I

| | Parts | % (dry) | parts (liquid) | mg/8 mg core |
|---|---|---|---|---|
| HPMC AS (Type MF) | 4.29 | 20 | 4.29 | 0.16 |
| Eudragit RS 30 D | 13.49 | 57.5 | 40.89 | 0.46 |
| Triethyl-citrate | 2.68 | 12.5 | 2.68 | 0.10 |
| Syloid 244 FP | 2.14 | 10 | 2.14 | 0.08 |
| Water. | q.s. | q.s. | 50.00 | |
| Total | 22.60 | 100 | 100.00 | 0.80 |

Optional Ratio: Eudragit RS:HPMC AS: 7.5:2.5
Coating layer: 5-20% (i.e. 10%) of core weight

### 2.2.3 Coatings Based on Ethylcellulose (+Pore Former)

The release rate of diffusion coats based on ethylcellulose might be controlled by the coating layer thickness (coating amount) and/or by the amount of hydrophilic coating compounds like plastisizers (Tritehylcitrate, PEG 4000, PEG 4000) or pigments/antisticking agents (like colloidal silicum dioxide, Syloid 244 FP) or by addition of pore forming polymers. Hydroxypropylmethylcellulose is a common known pore former to be combined with ethylcellulose applied from organic coating solution or in combination with Aquacoat ECD dispersions (30% aqueous dispersion of ethylcellulose). The ratio of ethylcellulose and pore former may vary between 95:5 and 50:50. Enteric polymers like Hydroxypropylmethyl-cellulose phthalate (HP 50) or Hydroxypropylmethylcellulose acetate succinate (aqoat) are also suitable pore formers to suppress the drug release in the acidic stomach and control the release in the intestinal juice with pH>5.5.

HP 50 can be combined with ethylcellulose coated from organic solution in the range of 5-50%. The coating layer applied is in the range of 5-30% of core weight, depending on the size and volume of the core pellet or minitablet.

EXAMPLE COATING J

| | parts | % (dry) | parts (liquid) | mg/8 mg core |
|---|---|---|---|---|
| Diffusion coat | | | | |
| Ethylcellulose | 6.75 | 75.00 | 6.75 | 0.60 |
| HPMC 3 cps | 0.75 | 8.33 | 0.75 | 0.07 |
| Aerosil 200 | 1.50 | 16.67 | 1.50 | 0.13 |
| Acetone | q.s. | q.s. | 45.50 | |
| Ethanol | q.s. | q.s. | 45.50 | |
| Total | 9.00 | 100.00 | 100.00 | 0.80 |

Ethylcellulose: HPMC 3 cps = 9:1
Coating layer: 5-20% (i.e. 10%) of core weight

EXAMPLE COATING K

| | parts (dry) | % (dry) | parts (liquid) | mg/8 mg core |
|---|---|---|---|---|
| Diffusion coat | | | | |
| Ethylcellulose | 6.00 | 66.67 | 6.00 | 0.80 |
| HP 50 | 1.50 | 16.67 | 1.50 | 0.20 |
| or Aqoat TYPE M | | | | |
| Aerosil 200 | 1.50 | 16.67 | 1.50 | 0.20 |
| Acetone | q.s. | q.s. | 45.50 | |
| Ethanol | q.s. | q.s. | 45.50 | |
| Total | 9.00 | 100.00 | 100.00 | 1.20 |

Ethylcellulose: HP 50 OR AQOAT = 8:2
Coating layer: 5-20% (i.e. 15%) of core weight

EXAMPLE COATING L

| | parts (dry) | % (dry) | parts (liquid) | mg/8 mg core |
|---|---|---|---|---|
| Diffusion coat | | | | |
| HPMC 3 cps | 1.33 | 11.16 | 1.33 | 0.09 |
| Aquacoat ECD | 8.37 | 70.22 | 25.11 | 0.56 |
| Triethyl-citrate or | 2.22 | 18.62 | 2.22 | 0.15 |
| Dibutylsebacate | | | | |
| Water. | q.s. | | 71.34 | |
| Total | 11.92 | 100.00 | 100.00 | 0.80 |

Ethylcellulose: HPMC = 8.5:1.5
Coating layer: 5-20% (i.e. 10%) of core weight

EXAMPLE COATING M

| | parts (dry) | % (dry) | parts (liquid) | mg/8 mg core |
|---|---|---|---|---|
| Diffusion coat | | | | |
| HPMC AS (MF) | 2.20 | 21.57 | 2.20 | 0.26 |
| Aquacoat ECD | 6.10 | 59.80 | 18.30 | 0.72 |
| Triethyl-citrate | 1.90 | 18.63 | 1.90 | 0.22 |
| Water. | q.s. | q.s. | 77.60 | |
| Total | 10.20 | 100.00 | 100.00 | 1.20 |

Ethylcellulose: AQOAT = 7:3
Coating layer: 5-20% (i.e. 15%) of core weight

### 2.3 Application of Diffusion Coatings on Multiparticulate Systems

The diffusion coat applied on a multiparticulate formulation (minitablets, pellets, granules, beads) is coated in fluidized bed equipment with Wurster principle or in a Mifflin type of equipment (turbojet) with a product temperature in the range of 28 to 45° C. It is proposed to cure (temper) the coat applied from aqueous dispersion after coating for 1-5 hours at 40° C. (Eudragit)-60° C. (Aquacoat) in a tray dryer or fluidized bed equipment. The final dosage form could be a stickpack or hard capsule filled with the multiparticulate formulation or a disintegrating tablet giving free the coated mulitparticulate pellets

In case of an organic solution, the plastisizer and polymers are dissolved in the organic solvent mixture and finally the antisticking agent is dispersed. For an aqueous dispersion the plastisizer is dissolved in water, the antisticking agent finely dispersed using a homogenizer. Finally the pre-prepared polymer dispersion (as commercially available) or predispersed in water is added to the plastisizer—antisticking agent—water mixture and stirred for some time before spraying.

### 3 Mixtures of Immediate Release and Modified Release Multiparticulates

### 3.1 Combination of IR and MR Pellets

Immediate and modified release pellets prepared according to process described in 2.1.2. (preparation of pellets) can be used as a combination by including them into the same capsule or sachet. For purposes of discussion, not limitation, the many combinations can include 10-90% of the drug loading in the immediate release formulation and 10-90% of the drug loading in the modified release formulation (90/10; 80/20; 70/30; 60/40; 50/50; 40/60; 30/70; 20/80; 10/90).

In addition, immediate release pellets prepared according to process described in 2.1.2. and modified release pellets prepared according to process described in 1.3 can be used as combination.

### 3.2 Combination of IR and MR Granules and Minitablets

Immediate and modified release granules or minitablets prepared according to process described in 2.1.1 (preparation of granules and minitablets) can be used as a combination by including them into the same capsule or sachet. For purposes of discussion, not limitation, the many combinations can include 10-90% of the drug loading in the immediate release formulation and 10-90% of the drug loading in the modified release formulation (90/10; 80/20; 70/30; 60/40; 50/50; 40/60; 30/70; 20/80; 10/90).

In addition, immediate release granules or minitables prepared according to process described in 2.1.1. and modified release granules or minitables prepared according to process described in 1.2 can be used as combination.

### 3.3 Combination of IR and MR Beads

Immediate and modified release beads prepared according to process described in 2.1.3 (preparation of beads) can be used as a combination by including them into the same capsule or sachet. For purposes of discussion, not limitation, the many combinations can include 10-90% of the drug loading in the immediate release formulation and 10-90% of the drug loading in the modified release formulation (90/10; 80/20; 70/30; 60/40; 50/50; 40/60; 30/70; 20/80; 10/90).

Additionally, modified release beads prepared according to process described in 2.1.3.1 and 2.1.3.2 can be further processed by dispensing them into a Wurster fluid bed coater and additionally coated with a drug solution previously prepared which is sprayed onto the seeds until the depletion. The beads are dried in the same conditions for 5 minutes. Additionally, a solution of hydroxypropyl methylcellulose (Opadry™) in purified water can be sprayed onto the seeds as a protective layer. On administration of such a dosage form the outer IR portion of the drug will dissolve completely at low pH in the stomach whereas the inner MR portion will completely diffuses from the pH-controlled systems in the small intestine.

### 4 Bilayer Tablets Comprising an Immediate Release and a Modified Release Layer or Two Modified Release Layers of Different Modified Release Profiles

Bi-layer tablets were prepared by filling the granules of the first layer in the die which were subsequently slightly compacted with a single punch press. Afterwards the granules of the second layer composition were filled on top of the slightly compressed tablet and compression force was being applied to manufacture a bilayer tablet.

EXAMPLE 19
bilayer tablet comprising an IR and a MR layer

| | Composition | Weight | % |
|---|---|---|---|
| Layer 1 MR | PTK787 (125 mg) | 167.5 mg | 23.1 |
| | Methocel K100LV | 125.0 mg | 17.2 |
| | Fumaric acid | 125.0 mg | 17.2 |
| | Lactose monohydrate | 20.0 mg | 2.8 |
| | Magnesium Stearate | 7.5 mg | 1.0 |
| | Aerosil | 5.0 mg | 0.7 |
| | | 450.0 mg | |
| Layer 2 IR | PTK787 (125 mg) | 167.5 mg | 23.1 |
| | Lactose monohydrate | 88.0 mg | 12.1 |
| | Hypromellose | 7.0 mg | 1.0 |
| | Croscarmellose sodium | 7.5 mg | 1.0 |
| | Magnesium Stearate | 5.0 mg | 0.7 |
| | | 275.0 mg | |
| Total | | 725.0 mg | 100.0 |

EXAMPLE 20
bilayer tablet comprising an IR and a MR layer

| | Composition | Weight | % |
|---|---|---|---|
| Layer 1 MR | PTK787 (125 mg) | 167.5 mg | 26.3 |
| | Methocel K100LV | 125.0 mg | 19.6 |
| | Fumaric acid | 37.5 mg | 5.9 |
| | Lactose monohydrate | 20.0 mg | 3.1 |
| | Magnesium Stearate | 7.5 mg | 1.2 |
| | Aerosil | 5.0 mg | 0.8 |
| | | 362.5 mg | |
| Layer 2 IR | PTK787 (125 mg) | 167.5 mg | 26.3 |
| | Lactose monohydrate | 88.0 mg | 13.8 |
| | Hypromellose | 7.0 mg | 1.1 |
| | Croscarmellose sodium | 7.5 mg | 1.2 |
| | Magnesium Stearate | 5.0 mg | 0.8 |
| | | 275.0 mg | |
| Total | | 637.5 mg | 100.0 |

### 5 In Vitro Dissolution Studies

The dissolution studies of matrix tablets are conducted using an USP I basket apparatus (Sotax A7). Dissolution tests are performed in triplicate using 1000 ml phosphate buffer (pH 6.8, SDS 0.2% w/V), at 37° C. and a rotational speed of 100 rpm. Minitablets are assessed using the same conditions with the exception that the dissolution studies were performed using an USP II paddle apparatus (Sotax A7). At predetermined intervals samples are withdrawn from the dissolution medium, filtered through a 0.45 μm membrane filters, and analyzed spectrophotometrically. Equivalent amounts of fresh buffer are added to maintain a constant dissolution volume.

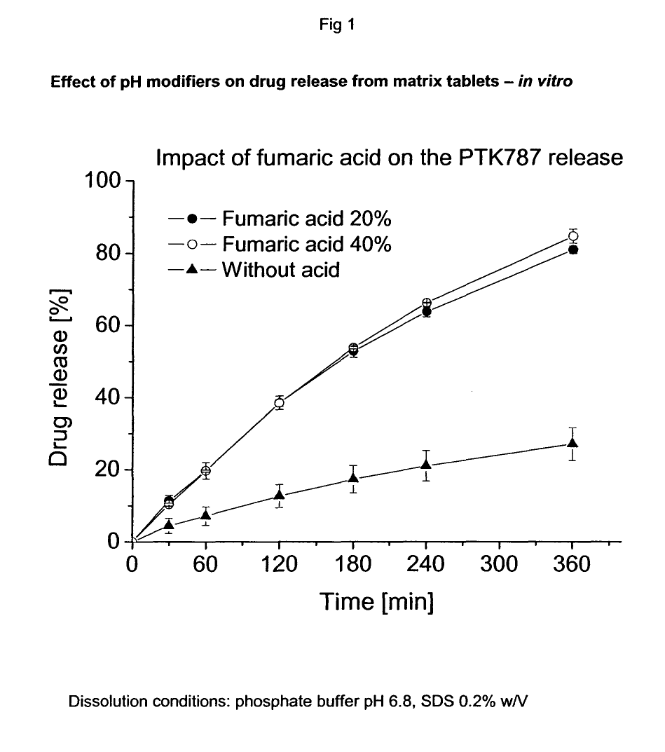

The incorporation of fumaric acid as pH modifier significantly enhances the PTK787 release at pH 6.8; consequently almost the entire drug is released after 6 hours (FIG. 1).

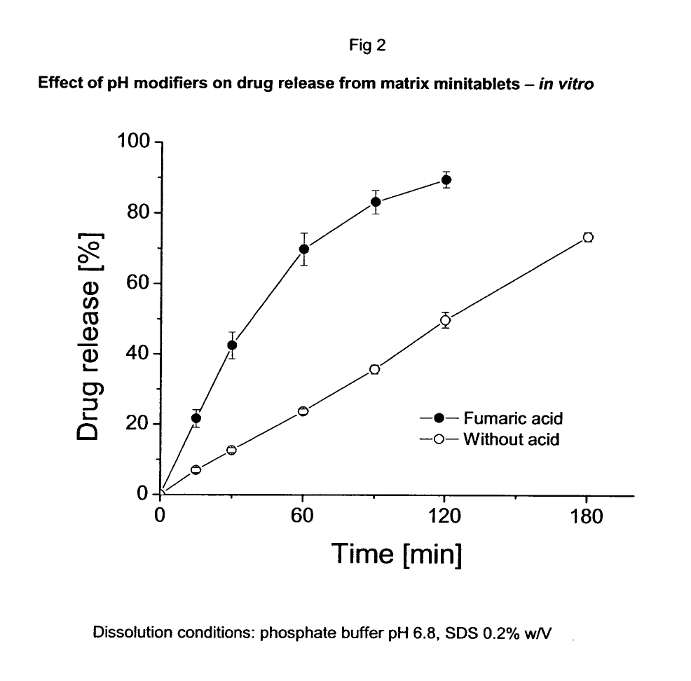

Consistent with this data, drug release from matrix minitablets with incorporated fumaric acid is markedly increased at pH 6.8 (FIG. 2).

### 6 In Vivo Absorption Study

The study is performed with six male beagle dogs fasted overnight for about 20 hours. The weight of the dogs ranges from 9.35 to 13.35 kg before the first drug administration. We use a two-block cross-over study design divided into a block of matrix tablets and one of matrix minitablets.

A ranitidine hydrochloride solution (50 mg/5 ml) diluted in a ratio of 1:1 with 5% glucose is injected intravenously as slow bolus within 2 min and 30 min prior to the administration of the (mini)-tablets. Two tablets or two capsules filled with minitablets (100 mg PTK787/dog) are administered orally deep into the throat followed by a rinse with 20 ml water gavages through a plastic syringe. Four hours after the administration of the tablet formulations the dogs are offered a standard dog chow of 300 g pellets. Free excess to water is allowed all the time. Blood samples of 2 ml are collected from the vena cephalica before (t=0) and after 0.25, 0.50, 0.75, 1, 1.5, 2, 3, 4, 6, 8, 10, and 24 hours post dose into heparinized syringes. The plasma is obtained after centrifugation for 10 min at 4° C.

The drug concentration in the plasma is determined using a HPLC MS/MS method.

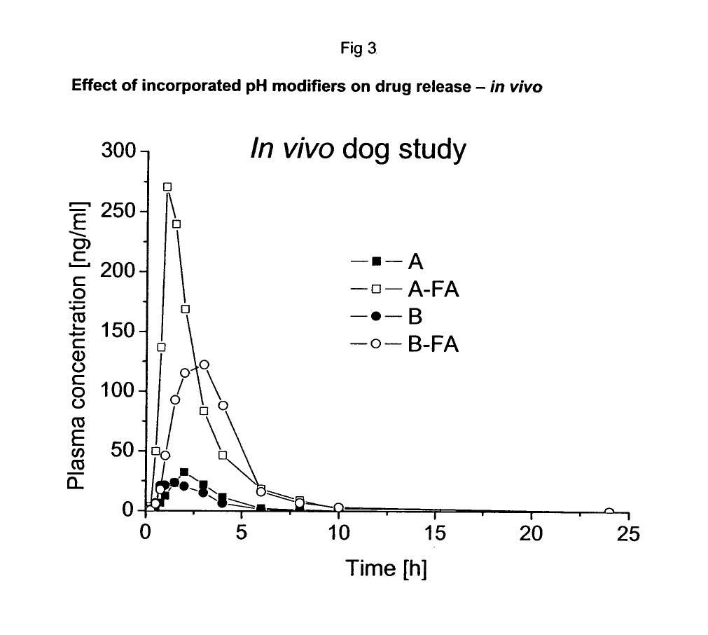

Incorporation of the pH modifier significantly enhances the in vitro and in vivo performance of PTK787 from matrix tablets and matrix minitablets. In case of the monolithic matrix tablet the AUC(0-24 h) increases approximately the 5-fold due to the presence of fumaric acid, and in case of the minitablets an 8-fold increase in the mean AUC(0-24 h) levels due to the pH-modifier (p<0.001) can be observed (FIG. 3).

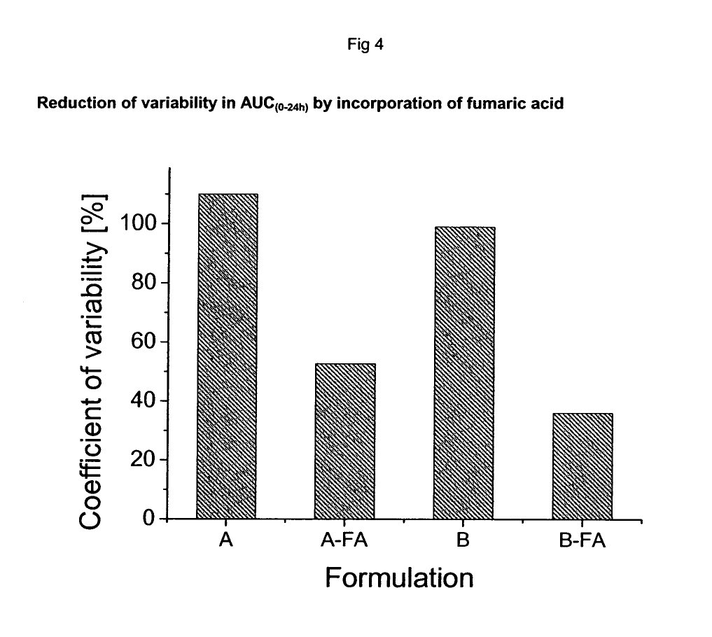

Additionally, the inclusion of a pH modifier distinctively reduced the inter-dog variability in terms of the coefficient variability (p<0.001) (FIG. 4).

The following table summarizes the mean pharmacokinetic parameters obtained from the monolithic tablets and minitablets.

Serum concentrations and pharmacokinetic parameters
Mean (n = 6) PK parameters of PTK787

| | Formulation | | | |
|---|---|---|---|---|
| | A | A-FA | B | B-FA |
| Dose (mg/dog) | 100.0 | 100.0 | 100.0 | 100.0 |
| tₘₐₓ | 1.7 | 2.4 | 1.4 | 1.2 |
| Cₘₐₓ | 34.9 | 158.3 | 35.7 | 298.8 |
| Cₘₐₓ/dose | 0.3 | 1.6 | 0.4 | 3.0 |
| AUC_{(0-24 h)} | 95.4 | 484.6 | 74.1 | 621.8 |
| AUC_{(0-24 h)}/dose | 1.0 | 4.8 | 0.7 | 6.2 |

Units: tmax [h]. Cmax [ng/mL]. Cmax/dose [(ng/mL)/(mg/kg/day)].
AUC(0-24 h) [h · ng/mL]. AUC(0-24 h)/dose [(h · ng/mL)/(mg/kg/day)].
A = Matrix tablet without fumaric acid according to Example 5;
A-FA = Matrix tablet with fumaric acid according to Example 3;
B = Matrix minitablets without fumaric acid according to Example 5;
B-FA = Matrix minitablets with fumaric acid according to Example 3.

1. A solid pharmaceutical composition comprising:
(i) 1-(4-chloroanilino)-4-(4-pyridylmethyl)phthalazine (Agent) or a pharmaceutically acceptable salt thereof;
(ii) a pH modifier.

2. The pharmaceutical composition according to claim 1 comprising:
(i) 1-(4-chloroanilino)-4-(4-pyridylmethyl)phthalazine (Agent) or a pharmaceutically acceptable salt thereof;
(ii) a pH modifier;
(iii) a polymer.

3. The pharmaceutical composition according to claim 1 comprising 1-(4-chloroanilino)-4-(4-pyridylmethyl)phthalazine succinate.

4. The pharmaceutical composition according to claim 1 wherein the pH modifier is an organic or inorganic chemical material that is able to release hydrogen ions and is pharmaceutically acceptable.

5. The pharmaceutical composition according to claim 4 wherein the pH modifier is selected from an organic acid, an acidic polymers, and a latent acid.

6. The pharmaceutical composition according to claim 5 wherein the pH modifier is selected from citric acid, fumaric acid, succininc acid, succinic acid anhydride, adipic acid, aspartic acid, glutamic acid, and maleic acid.

7. The pharmaceutical composition according to claim 6 wherein the pH modifier is fumaric acid.

8. The pharmaceutical composition according to claim 6 wherein the pH modifier is succinic acid or succinic acid anhydride.

9. The pharmaceutical composition according to claim 5 wherein the pH modifier is an polymeric organic acid containing a linear backbone with acidic groups, or a branched backbone with acidic groups or mixtures thereof.

10. The pharmaceutical composition according to claim 1 wherein the weight/weight ratio of pH modifier to Agent is between 0.01:1 and 10:1.

11. The pharmaceutical composition according to claim 10 wherein the weight/weight ratio of pH modifier to Agent is between 0.5:1 and 2:1

12. The pharmaceutical composition according to claim 10 wherein the weight/weight ratio of pH modifier to Agent is about 1:1.

13. Use of Agent and excipients as defined in claim 1 for the preparation of a medicament for the treatment of patients with disorders associated with deregulated angiogenesis.

14. A method of orally administering Agent, e.g., for the treatment of disorders associated with deregulated angiogenesis, said method comprising orally administering to a patient in need of Agent therapy a pharmaceutical composition according to claim 1.
